(19) United States
(12) Patent Application Publication
(10) Pub. No.: US 2016/0262808 A1
(43) Pub. Date: Sep. 15, 2016

(54) SURGICAL INSTRUMENT AND METHOD

(71) Applicant: Warsaw Orthopedic, Inc., Warsaw, IN (US)

(72) Inventors: Nicholas M. Benson, Cordova, TN (US); Jason M. May, Cordova, TN (US); William A. Rezach, Atoka, TN (US)

(21) Appl. No.: 14/645,189

(22) Filed: Mar. 11, 2015

(51) Int. Cl. A61B 17/70

(52) U.S. Cl. CPC A61B 17/7076

(57) ABSTRACT

The surgical instrument includes a first arm that is connectable with a fastener. A second arm is connectable with the fastener. The arms are relatively rotatable between a first position and a second dosed position. A lock is engageable with the arms in the dosed position to fix the fastener with the arms. Systems and methods are disclosed.

### TECHNICAL HELD

The present disclosure generally relates to medical devices for the treatment of spinal disorders, and more particularly to a surgical instrument and method for correction of a spine disorder.

### BACKGROUND

Spinal disorders such as degenerative disc disease, disc herniation, osteoporosis, spondylolisthesis, stenosis, scoliosis and other curvature abnormalities, kyphosis, tumor, and fracture may result from factors including trauma, disease and degenerative conditions caused by injury and aging. Spinal disorders typically result in symptoms including pain, nerve damage, and partial or complete loss of mobility.

Non-surgical treatments, such as medication, rehabilitation and exercise can be effective, however, may fail to relieve the symptoms associated with these disorders. Surgical treatment of these spinal disorders includes correction, fusion, fixation, discectomy, laminectomy and implantable prosthetics. As part of these surgical treatments, spinal constructs, which include implants such as bone fasteners, connectors, plates and vertebral rods are often used to provide stability to a treated region. These implants can redirect stresses away from a damaged or defective region while healing takes place to restore proper alignment and generally support the vertebral members. Surgical instruments are employed, for example, to engage the fasteners for attachment to the exterior of two or more vertebral members. This disclosure describes an improvement over these prior art technologies.

### SUMMARY

In one embodiment, a surgical instrument is provided. The surgical instrument includes a first arm that is connectable with a fastener. A second arm is connectable with the fastener. The arms are relatively rotatable between a first position and a second dosed position. A lock is engageable with the arms in the dosed position to fix the fastener with the arms. In some embodiments, systems and methods are disclosed.

### BRIEF DESCRIPTION OF THE DRAWINGS

The present disclosure will become more readily apparent from he specific description accompanied by the following drawings, in which:

FIG. 1 is a side view of components of one embodiment of a spinal implant system in accordance with the principles of the present disclosure;

FIG. 2 is a side view of components of one embodiment of a spinal implant system in accordance with the principles of the present disclosure;

FIG. 3 is a side view of the components shown in FIG. 2;

FIG. 4 is a break away cross section view of the components shown in FIG. 3;

FIG. 5 is a side view of components of one embodiment of a spinal implant system in accordance with the principles of the present disclosure; and

FIG. 6 is a side view of components shown in FIG. 5.

### DETAILED DESCRIPTION

The exemplary embodiments of the system and related methods of use disclosed are discussed in terms of medical devices for the treatment of musculoskeletal disorders and more particularly, in terms of a surgical system and method for correction of a spine disorder. In one embodiment, the surgical implant system includes a bone fastener that allows the head to be captured and retained by an implant support and/or surgical instrument, such as, for example, an extender.

In some embodiments, the extender includes a latch for disposing the extender between an open position and a dosed position. In some embodiments, the extender includes a latch retaining element for engaging the latch. In some embodiments, the extender includes a latch retaining element configured to provide interference such that arms of the extender can flex during attachment to a receiver of a bone screw, such as, for example, a tulip head.

In some embodiments, the extender includes a cavity configured for disposal of an implant, such as, for example, a bone screw. In some embodiments, the extender includes arms that move, translate, rotate and/or flex to accommodate a pin engaging retaining element of a bone screw such that a secure connection is provided with the screw.

In some embodiments, the extender includes a mating element, such as, for example, a threaded engagement member that is configured for engagement with a mating element, such as, for example, a threaded portion of a removable handle attachment. In some embodiments, the mating elements can guide the handle into alignment with the extender. This configuration can prevent cross threading of threaded mating elements. In some embodiments, the extender can be connected with various surgical instruments, such as, for example, rod reducers, derotation bridges and/or other vertebral column manipulation instruments and/or employed with vertebral column manipulation procedures. In some embodiments, the extender can be employed for engagement with a tulip head of a bone screw. In some embodiments, the extender can be connected and employed with a compression rack to compress or distract and restore curvature of a spine, and/or restore vertebral body height and lordosis. For example, the system can include a handle that connects with the extender and a compression rack.

In some embodiments, one or all of the components of the system may be disposable, peel pack and/or pre packed sterile devices. One or all of the components of the system may be reusable. The system may be configured as a kit with multiple sized and configured components.

In some embodiments, the present disclosure may be employed to treat spinal disorders such as, for example, degenerative disc disease, disc herniation, osteoporosis, spondylolisthesis, stenosis, scoliosis and other curvature abnormalities, kyphosis, tumor and fractures. In some embodiments, the present disclosure may be employed with other osteal and bone related applications, including those associated with diagnostics and therapeutics. In some embodiments, the disclosed system may be alternatively employed in a surgical treatment with a patient in a prone or supine position, and/or employ various surgical approaches to the spine, including anterior, posterior, posterior mid-line, direct lateral, postero-lateral, and/or antero lateral approaches, and in other body regions. The present disclosure may also be alternatively employed with procedures for treating the lumbar, cervical, thoracic and pelvic regions of a spinal column. The system and methods of the present disclosure may also be used on animals, bone models and other non-living substrates, such as, for example, in training, testing and demonstration.

The present disclosure may be understood more readily by reference to the following detailed description of the disclosure taken in connection with the accompanying drawing figures, which form a part of this disclosure. It is to be understood that this disclosure is not limited to the specific devices, methods, conditions or parameters described and/or shown herein, and that the terminology used herein is for the purpose of describing particular embodiments by way of example only and is not intended to be limiting of the claimed disclosure. Also, in some embodiments, as used in the specification and including the appended claims, the singular forms “a,” “an,” and “the” include the plural, and reference to a particular numerical value includes at least that particular value, unless the context clearly dictates otherwise. Ranges may be expressed herein as from “about” or “approximately” one particular value and/or to “about” or “approximately” another particular value. When such a range is expressed, another embodiment includes from the one particular value and/or to the other particular value. Similarly, when values are expressed as approximations, by use of the antecedent “about,” it will be understood that the particular value forms another embodiment. It is also understood that all spatial references, such as, for example, horizontal, vertical, top, upper, lower, bottom, left and right, are for illustrative purposes only and can be varied within the scope of the disclosure. For example, the references “upper” and “lower” are relative and used only in the context to the other, and are not necessarily “superior” and “inferior”.

Further, as used in the specification and including the appended claims, “treating” or “treatment” of a disease or condition refers to performing a procedure that may include administering one or more drugs to a patient (human, normal or otherwise or other mammal), in an effort to alleviate signs or symptoms of the disease or condition. Alleviation can occur prior to signs or symptoms of the disease or condition appearing, as well as after their appearance. Thus, treating or treatment includes preventing or prevention of disease or undesirable condition (e.g., preventing the disease from occurring in a patient, who may be predisposed to the disease but has not yet been diagnosed as having it). In addition, treating or treatment does not require complete alleviation of signs or symptoms, does not require a cure, and specifically includes procedures that have only a marginal effect on the patient. Treatment can include inhibiting the disease, e.g., arresting its development, or relieving the disease, e.g., causing regression of the disease. For example, treatment can include reducing acute or chronic inflammation; alleviating pain and mitigating and inducing re-growth of new ligament, bone and other tissues; as an adjunct in surgery; and/or any repair procedure. Also, as used in the specification and including the appended claims, the term “tissue” includes soft tissue, vessels, ligaments, tendons, cartilage and/or bone unless specifically referred to otherwise.

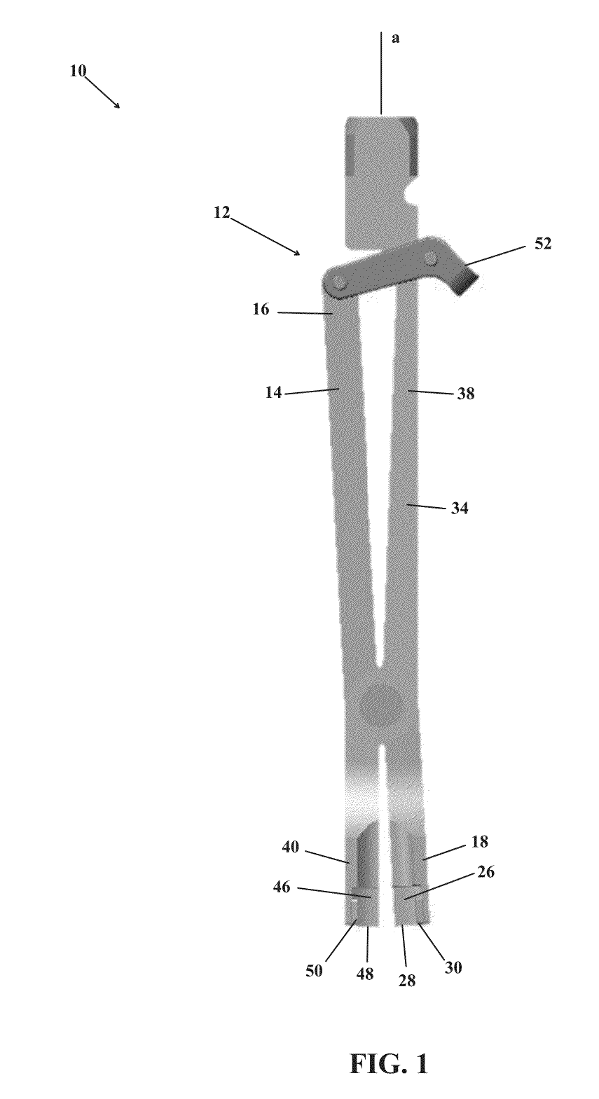

The following discussion includes a description of a system in accordance with the principles of the present disclosure. Alternate embodiments are also disclosed. Reference is made in detail to the exemplary embodiments of the present disclosure, which are illustrated in the accompanying figures. Turning to FIG. 1, there are illustrated components of a system, such as, for example, a spinal implant system 10,

The components of system 10 can be fabricated from biologically acceptable materials suitable for medical applications, including metals, synthetic polymers, ceramics, bone material, tissue and/or their composites, depending on the particular application and/or preference of a medical practitioner. For example, the components of system 10, individually or collectively, can be fabricated from materials such as stainless steel alloys, aluminum, commercially pure titanium, titanium alloys, Grade 5 titanium, super-elastic titanium alloys, cobalt-chrome alloys, stainless steel alloys, superelastic metallic alloys (e.g., Nitinol, super elasto-plastic metals, such as GUM METAL® manufactured by Toyota Material Incorporated of Japan), ceramics and composites thereof such as calcium phosphate (e.g., SKELITE™ manufactured by Biologix Inc.), thermoplastics such as polyaryletherketone (PAEK) including polyetheretherketone (PEEK), polyetherketoneketone (PEKK) and polyetherketone (PEK), carbon-PEEK composites, PEEK-BaSO₄ polymeric rubbers, polyethylene terephthalate (PET), fabric, silicone, polyurethane, silicone-polyurethane copolymers, polymeric rubbers, polyolefin rubbers, hydrogels, semi-rigid and rigid materials, elastomers, rubbers, thermoplastic elastomers, thermoset elastomers, elastomeric composites, rigid polymers including polyphenylene, polyamide, polyimide, polyetherimide, polyethylene, epoxy, bone material including autograft, allograft, xenograft or transgenic cortical and/or corticocancellous bone, and tissue growth or differentiation factors, partially resorbable materials, such as, for example, composites of metals and calcium-based ceramics, composites of PEEK and calcium based ceramics, composites of PEEK with resorbable polymers, totally resorbable materials, such as, for example, calcium based ceramics such as calcium phosphate, tri-calcium phosphate (TCP), hydroxyapatite (HA)-TCP, calcium sulfate, or other resorbable polymers such as polyaetide, polyglycolide, polytyrosine carbonate, polycaroplaetohe and their combinations. Various components of system 10 may have material composites, including the above materials, to achieve various desired characteristics such as strength, rigidity, elasticity, compliance, biomechanical performance, durability and radiolucency or imaging preference. The components of system 10, individually or collectively, may also be fabricated from a heterogeneous material such as a combination of two or more of the above-described materials. The components of system 10 may be monolithically formed, integrally connected or include fastening elements and/or instruments, as described herein.

System 10 includes an implant support and/or surgical instrument, such as, for example, an extender 12. Extender 12 includes an arm 14 that extends between an end 16 and an end 18. In some embodiments, the cross section and/or overall configuration of arm 14 may be variously configured, such as, for example, round, oval, oblong, square, rectangular, polygonal, irregular, uniform, non-uniform, offset, staggered, tapered, consistent or variable. In some embodiments, arm 14 may include an outer gripping surface configured for gripping by a hand of a practitioner. In some embodiments, the gripping surface may be, such as, for example, rough, arcuate, undulating, mesh, porous, semi-porous, dimpled and/or textured.

Arm 14 includes a member 20 disposed adjacent end 16 and a member 22 disposed adjacent end 18. Members 20, 22 are disposed in a substantially offset alignment. Member 20 is integrally connected or monolithically formed with member 22 such that members 20, 22 simultaneously rotate relative to a second arm and relative to a longitudinal axis a of extender 12.

End 18 is configured for connection with a fastener, such as, for example, a bone screw 24. End 18 defines an inner surface 26 that defines a cavity 28 configured for disposal of bone screw 24. Surface 26 includes one or more mating elements, such as, for example, a projection 30 that engages and removably locks with one or more mating elements of an arm 32 of a receiver of bone screw 24, as described herein. Projection 30 extends into cavity 28 to engage bone screw 24 for retaining bone screw 24 with member 22. In some embodiments, all or only a portion of surface 26 may have alternate surface configurations to enhance fixation with bone screw 24, such as, for example, dimpled and/or textured. In some embodiments, projection 30 may include a nail configuration, raised dements and/or spikes to facilitate engagement with bone screw 24.

An arm 34 is connected to arm 14 via a pivot, which includes a hinge 36. Hinge 36 is centrally disposed and configured to facilitate rotation of arm 34 relative to arm 14, and relative to axis a. In some embodiments, hinge 36 may be variously configured such as, for example, pin, post, screw, living hinge, ratchet and/or concentric parts. Arm 34 extends between an end 38 and an end 40. In some embodiments, the cross section and/or overall configuration of arm 34 may be variously configured, such as, for example, round, oval, oblong, square, rectangular, polygonal, irregular, uniform, non-uniform, offset, staggered, tapered, consistent or variable. In some embodiments, arm 34 may include an outer gripping surface configured for gripping by a hand of a practitioner. In some embodiments, the gripping surface may be, such as, for example, those alternatives described herein,

Arm 34 includes a member 42 disposed adjacent end 38 and a member 44 disposed adjacent end 40. Members 42, 44 are disposed in a substantially offset alignment. Member 42 is integrally connected or monolithically formed with member 44 such that members 42, 44 simultaneously rotate relative to arm 14.

End 40 is configured for connection with a fastener, such as, for example, bone screw 24. End 40 defines an inner surface 46 that defines a cavity 48 configured for disposal of bone screw 24. Surface 46 includes one or more mating elements, such as, for example, a projection 50 that engages and removably locks with one or more mating elements of arm 32 of bone screw 24, as described herein. Projection 50 extends into cavity 48 to engage bone screw 24 for retaining bone screw 24 with member 44. In some embodiments, all or only a portion of surface 46 may have alternate surface configurations to enhance fixation with bone screw 24, such as, for example, dimpled and/or textured. In some embodiments, projection 50 may include a nail configuration, raised elements and/or spikes to facilitate engagement with bone screw 24.

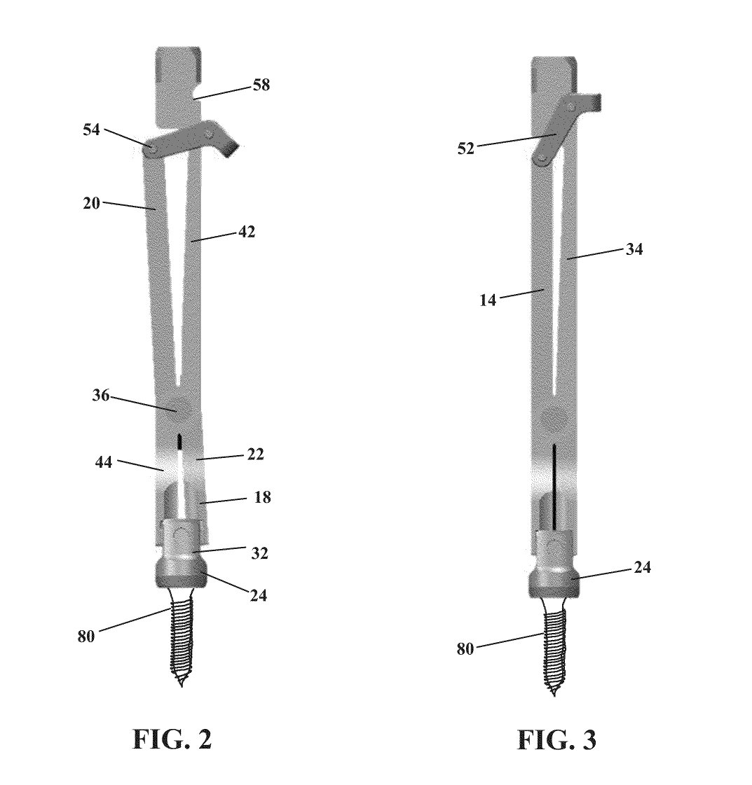

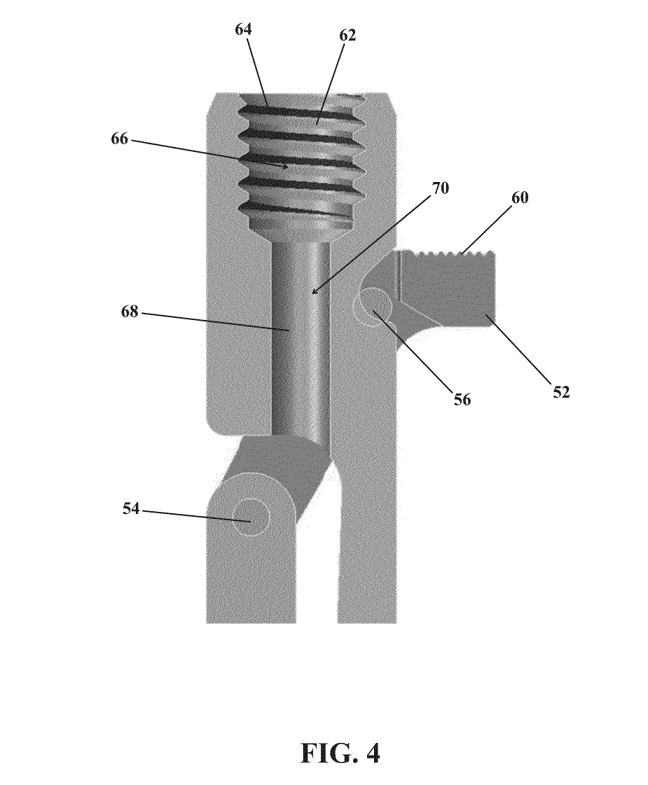

Arms 14 and 34 are configured for relative rotation and pivotal movement relative to axis a to move between a first open position, as shown in FIG. 2, and a second dosed position, as shown in FIG. 3, to engage and/or capture bone screw 24 for releasable locking and/or fixation therewith. Extender 12 includes a lock, such as, for example, a latch 52 connected to end 16 via a pivot, which includes a hinge 54. Hinge 54 is configured to facilitate rotation of latch 52 relative to member 20. In some embodiments, hinge 54 may be variously configured such as, for example, pin, post, screw, living hinge, ratchet and/or concentric parts. Latch 52 includes a mating element, such as, for example, a pin 56 engageable with a mating element, such as, for example, a retaining groove 58, which includes a surface of end 38 that defines a cavity for disposal of pin 56. Groove 58 includes a ledge surface that engages pin 56 in an interference fixation to lock extender 12 in a dosed position, as shown in FIG. 4, for example, with bone screw 24, as shown in FIG. 3 and described herein. In some embodiments, latch 52 includes a gripping surface 60 configured for gripping by a hand of a practitioner. In some embodiments, gripping surface 60 may be, such as, for example, those alternatives described herein.

In use, arms 14, 34 are disposed in an open position, as shown in FIG. 2, and bone screw 24 is aligned with extender 12 such that arm 32 is disposed with cavities 28, 48. Arms 14, 34 are relatively rotated and pivoted relative to axis a to move from the open position to a second closed position, as shown in FIG. 3. Arms 14, 34 flex to engage and/or capture bone screw 24 such that the mating elements, as described herein, of bone screw 24 and ends 18, 40 releasably lock. In the closed position, latch 52 is rotated relative to member 20 such that pin 56 is disposed in groove 58 and engages the surface of groove 58 in an interference fixation to dispose extender 12 with bone screw 24 in a locked configuration. Latch 52 can be rotated in an opposite direction to overcome the interference fixation such that pin 56 exits groove 58 to dispose extender 12 in a nonlocking configuration such that bone screw 24 is releasable from ends 18, 40.

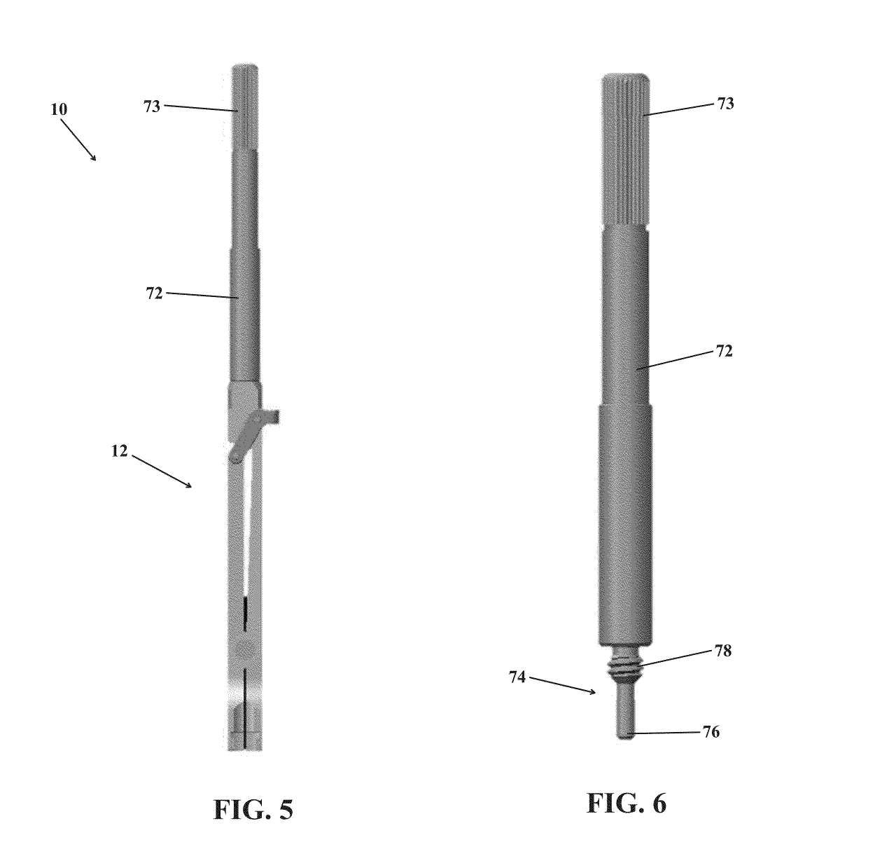

In some embodiments, as shown in FIGS. 4-6, member 42 includes a mating surface, such as, for example, an inner surface 62. Surface 62 includes a threaded surface 64 that defines a threaded cavity 66. Surface 62 also includes an even surface 68 that defines an alignment cavity 70. Member 42 is connectable with a handle 72, as shown in AGS. 5 and 6. Handle 72 includes a gripping surface 73 and is configured for manipulation and/or maneuvering of extender 12. In some embodiments, handle 72 may be utilized to facilitate engagement of bone screw 24 with tissue. Handle 72 may have various surface configurations, such as, for example, smooth, rough, arcuate, undulating, porous, semi-porous, dimpled, polished and/or textured.

Handle 72 includes an extension 74 including a protrusion, such as, for example, a guide 76 and a mating surface, such as, for example, a threaded surface 78. Guide 76 is aligned and disposable with cavity 70 for alignment and mating of handle 72 with extender 12. Surface 78 is threaded with surface 64 such that handle 72 is removable attached with extender 12.

In some embodiments, all or only a portion of handle 72, end 14 and/or end 38 are configured for connection with a surgical instrument, such as, for example, compression and/or distraction instruments, such as, for example, a compression rack to correct a spinal disorder, such as, for example, curvature abnormalities, trauma and/or fracture of vertebrae, which may include a sagittal deformity and requires compression and/or distraction. In some embodiments, handle 72 and/or extender 12 can be connected with various surgical instruments, such as, for example, rod reducers, derotation bridges and/or other vertebral column manipulation instruments and/or employed with vertebral column manipulation procedures. In some embodiments, handle 72 and/or extender 12 can be connected with a surgical instrument and employed to compress or distract and restore curvature of a spine, and/or restore vertebral body height and lordosis.

System 10 includes a fastener, such as, for example, a bone screw 24. Bone screw 24 is configured for attachment with one or more extenders 12 and includes an elongated shaft configured for penetrating tissue. Shaft 80 has a cylindrical cross section configuration and includes an outer surface having an external thread form. In some embodiments, the thread form may include a single thread turn or a plurality of discrete threads. In some embodiments, other engaging structures may be disposed on shaft 80, such as, for example, a nail configuration, barbs, expanding elements, raised elements and/or spikes to facilitate engagement of shaft 80 with tissue, such as, for example, vertebrae.

In some embodiments, all or only a portion of shaft 80 may have alternate cross section configurations, such as, for example, oval, oblong, triangular, square, polygonal, irregular, uniform, non-uniform, offset, staggered, undulating, arcuate, variable and/or tapered. In some embodiments, the outer surface may include one or a plurality of openings. In some embodiments, all or only a portion of the outer surface may have alternate surface configurations to enhance fixation with tissue such as, for example, rough, arcuate, undulating, mesh, porous, semi-porous, dimpled and/or textured. In some embodiments, all or only a portion of shaft 80 may be disposed at alternate orientations, relative to a longitudinal axis of bone fastener 24, such as, for example, transverse, perpendicular and/or other angular orientations such as acute or obtuse, co-axial and/or may be offset or staggered. In some embodiments, all or only a portion of shaft 80 may be cannulated.

In assembly, operation and use, spinal implant system 10, similar to the systems and methods described herein, is employed with a surgical procedure for treatment of a spinal disorder affecting a section of a spine of a patient, as discussed herein. For example, system 10 can be used with a surgical procedure for treatment of a condition or injury of an affected section of the spine including vertebrae. In some embodiments, one or all of the components of system 10 can be delivered as a pre-assembled device or can be assembled in situ. System 10 may be completely or partially revised, removed or replaced.

For example, system 10 can be employed with a surgical treatment of an applicable condition or injury of an affected section of a spinal column and adjacent areas within a body, such as, for example, vertebrae. In some embodiments, system 10 may be employed with one or a plurality of vertebra. To treat a selected section of the vertebrae, a medical practitioner obtains access to a surgical site including the vertebrae in any appropriate manner, such as through incision and retraction of tissues. In some embodiments, system 10 can be used in any existing surgical method or technique including open surgery, mini-open surgery, minimally invasive surgery including percutaneous surgical implantation, whereby the vertebrae are accessed through a mini-incision, or sleeve that provides a protected passageway to the area. Once access to the surgical site is obtained, the particular surgical procedure can be performed for treating the spine disorder.

An incision is made in the body of a patient and a cutting instrument (not shown) creates a surgical pathway for delivery of implantable components of system 10 such as, for example, bone screw 24, as shown in FIG. 2. A preparation instrument (not shown) can be employed to prepare tissue surfaces of vertebrae, as well as for aspiration and irrigation of a surgical region.

Pilot holes or the like are made in selected vertebrae for receiving one or more of shafts 80 of bone screws 24. A driver (not shown) is disposed adjacent vertebrae at a surgical site and is manipulated to drive, torque, insert or otherwise connect shafts 80 of bone screws 24 adjacent vertebrae. One or more extenders 12 are delivered to the surgical site adjacent vertebrae and oriented for manipulation, alignment and capture of bone screws 24, as described herein with regard to FIGS. 1-6.

In some embodiments, spinal implant system 10 includes one or more fasteners for attaching a spinal construct with tissue, as described herein. In some embodiments, the fasteners may be engaged with tissue in various orientations, such as, for example, series, parallel, offset, staggered and/or alternate vertebral levels. In some embodiments, one or more of the fasteners may comprise multi-axial screws, sagittal angulation screws, pedicle screws, facet screws, tissue penetrating screws, conventional screws and/or expanding screws.

In one embodiment, spinal implant system 10 includes an agent, which may be disposed, packed, coated or layered within, on or about the components and/or surfaces of spinal implant system 10. In some embodiments, the agent may include bone growth promoting material, such as, for example, bone graft to enhance fixation of the components and/or surfaces of spinal implant system 10 with vertebrae. In some embodiments, the agent may include one or a plurality of therapeutic agents and/or pharmacological agents for release, including sustained release, to treat, for example, pain, inflammation and degeneration.

Upon completion of a procedure, extenders 12, surgical instruments and/or tools, assemblies and non-implanted components of system 10 are removed and the incision(s) are closed. One or more of the components of system 10 can be made of radiolucent materials such as polymers. Radiomarkers may be included for identification under x-ray, fluoroscopy, CT or other imaging techniques. In some embodiments, the use of surgical navigation, microsurgical and image guided technologies may be employed to access, view and repair spinal deterioration or damage, with the aid of system 10. In some embodiments, system 10 may include one or a plurality of plates, connectors and/or bone fasteners for use with a single vertebral level or a plurality of vertebral levels.

it will be understood that various modifications may be made to the embodiments disclosed herein. Therefore, the above description should not be construed as limiting, but merely as exemplification of the various embodiments. Those skilled in the art will envision other modifications within the scope and spirit of the claims appended hereto.

What is claimed is:

1. A surgical instrument comprising:
a first arm being connectable with a fastener;
a second arm being connectable with the fastener, the arms being relatively rotatable between a first position and a second closed position; and
a lock engageable with the arms in the closed position to fix the fastener with the arms.

2. A surgical instrument as recited in claim 1, wherein the arms are connected by a hinge.

3. A surgical instrument as recited in claim 1, wherein the lock is connected to one of the arms via a hinge such that the latch is rotatable relative to the arm.

4. A surgical instrument as recited in claim 1, wherein the lock includes a mating element engageable with a mating element of one of the arms.

4. cal instrument as recited in claim 4, wherein the mating element of the lock includes a pin.

6. A surgical instrument as recited in claim 4, wherein the mating element of the arm includes a groove.

7. A surgical instrument as recited claim 1, wherein the lock includes a mating element engageable with a mating element of one of the arms in an interference fixation.

8. A surgical instrument as recited in claim 1, further comprising a removable handle.

9. A surgical instrument as recited in claim 8, wherein the handle includes a guide configured to align the handle with at least one of the arms.

10. A surgical instrument as recited in claim 1, further comprising a removable handle including a mating surface engageable with a mating surface of one of the arms.

11. A surgical instrument as recited in claim 1, wherein each of the arms define an implant cavity and at least one projection extending therein that are releasably engageable with a fastener.
